(19) United States
(12) Patent Application Publication
(10) Pub. No.: US 2014/0343624 A1
(43) Pub. Date: Nov. 20, 2014

(54) NEUROMODULATION OF SUBCELLULAR STRUCTURES WITHIN THE DORSAL ROOT GANGLION

(71) Applicant: SPINAL MODULATIONS, INC., Menlo Park, CA (US)

(72) Inventor: Jeffery M. Kramer, Menlo Park, CA (US)

(21) Appl. No.: 14/362,543

(22) PCT Filed: Dec. 7, 2012
(86) PCT No.: PCT/US2012/068576

§ 371 Date: Jun. 3, 2014

Related U.S. Application Data
(60) Provisional application No. 61/568,093, filed on Dec. 7, 2011.

(51) Int. Cl. A61N 1/36; G01N 33/483

(52) U.S. Cl. CPC A61N 1/36071; A61N 1/36178; G01N 33/4833
USPC 607/46; 607/117; 607/72; 607/76; 73/866.4

(57) ABSTRACT

Devices, systems and methods are provided for the targeted treatment of abnormal sensory conditions. In such conditions, physical stimuli is transduced into neuronal impulses that are subsequently transmitted to the central nervous system for processing. Such transduction is achieved by primary sensory neurons in the dorsal root ganglions. Subcellular structures on primary sensory neurons can significantly modulate the function of these neurons, thereby affecting the transduction and reducing the abnormal sensory experiences. Thus, devices, systems and methods are provided for neuromodulating subcellular structures on primary sensory neurons of the dorsal root ganglions.

### CROSS-REFERENCES TO RELATED APPLICATION

This application claims priority under 35 U.S.C. 119(e) to U.S. Provisional Patent Application Ser. No. 61/568,093 filed on Dec. 7, 2012, which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Pain of any type is the most common reason for physician consultation in the United States, prompting half of all Americans to seek medical care annually. It is a major symptom in many medical conditions, significantly interfering with a person's quality of life and general functioning. Diagnosis is based on characterizing pain in various ways, according to duration, intensity, type (dull, burning, throbbing or stabbing), source, or location in body. Usually if pain stops without treatment or responds to simple measures such as resting or taking an analgesic, it is then called ‘acute’ pain. But it may also become intractable and develop into a condition called chronic pain in which pain is no longer considered a symptom but an illness by itself.

The application of specific electrical energy to the spinal cord for the purpose of managing pain has been actively practiced since the 1960s. It is known that application of an electrical field to spinal nervous tissue can effectively mask certain types of pain transmitted from regions of the body associated with the stimulated nervous tissue. Such masking is known as paresthesia, a subjective sensation of numbness or tingling in the afflicted bodily regions. Such electrical stimulation of the spinal cord, once known as dorsal column stimulation, is now referred to as spinal cord stimulation or SCS.

Conventional SCS systems include an implantable power source or implantable pulse generator (IPG) and an implantable lead. Such IPGs are similar in size and weight to cardiac pacemakers and are typically implanted in the buttocks or abdomen of a patient P. Using fluoroscopy, the lead is implanted into the epidural space of the spinal column and positioned against the dura layer of the spinal cord. The lead is implanted either through the skin via an epidural needle (for percutaneous leads) or directly and surgically through a mini laminotomy operation (for paddle leads or percutaneous leads). A laminotomy is a neurosurgical procedure that removes part of a lamina of the vertebral arch. The laminotomy creates an opening in the bone large enough to pass one or more leads through.

Implantation of a percutaneous lead typically involves an incision over the low back area (for control of back and leg pain) or over the upper back and neck area (for pain in the arms). An epidural needle is placed through the incision into the epidural space and the lead is advanced and steered over the spinal cord until it reaches the area of the spinal cord that, when electrically stimulated, produces a tingling sensation (paresthesia) that covers the patient's painful area. To locate this area, the lead is moved and turned on and off while the patient provides feedback about stimulation coverage. Because the patient participates in this operation and directs the operator to the correct area of the spinal cord, the procedure is performed with conscious sedation.

Although such SCS systems have effectively relieved pain in some patients, these systems have a number of drawbacks. To begin, the lead is positioned upon the spinal dura layer so that the electrodes stimulate a wide portion of the spinal cord and associated spinal nervous tissue. Significant energy is utilized to penetrate the dura layer and cerebral spinal fluid to activate fibers in the spinal column extending within the posterior side of the spinal cord to the dorsal roots. Sensory spinal nervous tissue, or nervous tissue from the dorsal nerve roots, transmit pain signals. Therefore, such stimulation is intended to block the transmission of pain signals to the brain with the production of a tingling sensation (paresthesia) that masks the patient's sensation of pain. However, excessive tingling may be considered undesirable. Further, the energy also typically penetrates the anterior side of the spinal cord, stimulating the ventral horns, and consequently the ventral roots extending within the anterior side of the spinal cord. Motor spinal nervous tissue, or nervous tissue from ventral nerve roots, transmits muscle/motor control signals. Therefore, electrical stimulation by the lead often causes undesirable stimulation of the motor nerves in addition to the sensory spinal nervous tissue. The result is undesirable muscle contraction.

Because the electrodes span several levels and because they stimulate medial to spinal root entry points, the generated stimulation energy stimulates or is applied to more than one type of nerve tissue on more than one level. Moreover, these and other conventional, non-specific stimulation systems also apply stimulation energy to the spinal cord and to other neural tissue beyond the intended stimulation targets. As used herein, non-specific stimulation refers to the fact that the stimulation energy is provided to multiple spinal levels including the nerves and the spinal cord generally and indiscriminately. This is the case even with the use of programmable electrode configurations wherein only a subset of the electrodes are used for stimulation. In fact, even if the epidural electrode is reduced in size to simply stimulate only one level, that electrode will apply stimulation energy non-specifically and indiscriminately (i.e. to many or all nerve fibers and other tissues) within the range of the applied energy.

Therefore, improved stimulation systems, devices and methods are desired that enable more precise and effective delivery of stimulation energy. At least some of these objectives will be met by the present invention.

### SUMMARY OF THE DISCLOSURE

The present invention provides methods, systems and devices for neuromodulation of a dorsal root ganglion. In a first aspect of the present invention, a method of neuromodulation is provided comprising positioning at least one electrode in proximity to a dorsal root ganglion, and energizing the at least one electrode so that an electric field is applied to the dorsal root ganglion in a manner which neuromodulates at least one subcellular structure on a primary sensory neuron within the dorsal root ganglion.

In some embodiments, neuromodulating the at least one subcellular structure comprises hyperpolarizing a cell membrane of the primary sensory neuron. In some embodiments, the subcellular structure comprises an ion channel of a cell membrane of the primary sensory neuron. Optionally, the ion channel comprises a potassium ion channel.

In some embodiments, neuromodulating the at least one subcellular structure comprises reducing cellular firing characteristics of the primary sensory neuron.

It may be appreciated that in some embodiments, the dorsal root ganglion is associated with an abnormal sensory condition of a patient and wherein neuromodulating the at least one subcellular structure reduces a symptom of the sensory condition. In some instances, the abnormal sensory condition comprises pain, puritis, dysthesias, phantom limb pain or a combination of these.

It may be appreciated that in some embodiments, the dorsal root ganglion is disposed within an in vitro model, wherein the method further comprises measuring an effect of the electric field on membrane excitability of the primary sensory neuron. In some instances, the measured effect indicates decreased membrane excitability.

In some embodiments, neuromodulating the at least one subcellular structure comprises modulating at least one t-junction. For example, modulating the at least one t-junction may comprise altering action potential conduction through the at least one t-junction. It may be appreciated that in some embodiments, the dorsal root ganglion is disposed within an in vitro model, wherein the method further comprises measuring amplitude of at least one train of action potentials through the at least one t-junction during and/or after neuromodulation. For example, measuring may comprise measuring a reduction in amplitude or measuring may comprise measuring a decrease in bursting behavior of the neuron associated with the t-junction.

In some embodiments, energizing the at least one electrode comprises providing an intermittent stimulation signal comprised of a series of bursts and inter-burst delays. For example, the bursts may have a frequency of approximately 4-1000 Hz. For example, the inter-burst delays may be approximately 4-1000 microseconds.

In a second aspect of the present invention, a method is provided of reducing excitability of a neuron within a dorsal root ganglion. In some embodiments, the method comprises applying an electric field to the dorsal root ganglion, wherein the electric field produces sufficient power to allow entry of calcium into the neuron to at least a level which activates calcium dependent potassium ion channels,whereby the potassium ion channels hyperpolarize the cell membrane making the neuron less excitable.

In some embodiments, applying the electric field to the dorsal root ganglion comprises positioning a lead having at least one electrode in proximity to the dorsal root ganglion within a patient so that at least one electrode provides the electric field. Optionally, positioning the lead comprises advancing the lead within an epidural space of the patient.

In some embodiments, the dorsal root ganglion is associated with an abnormal sensory condition of a patient, wherein making the neuron less excitable reduces symptoms of the sensory condition.

In some embodiments, applying the electric field to the dorsal root ganglion comprises positioning at least one electrode near the dorsal root ganglion, wherein the dorsal root has been explanted.

In a third aspect of the present invention, a method is provided of suppressing action potential firing in a sensory neuron within a dorsal root ganglion. In some embodiments, the method comprises applying an electric field to the dorsal root ganglion so that the electric field neuromodulates a t-junction associated with the sensory neuron in a manner which reduces action potential conduction through the t-junction.

In some embodiments, applying the electric field to the dorsal root ganglion comprises positioning a lead having at least one electrode in proximity to the dorsal root ganglion within a patient so that at least one electrode provides the electric field. Optionally, the lead comprises advancing the lead within an epidural space of the patient.

In some embodiments, the dorsal root ganglion is associated with an abnormal sensory condition of the patient, wherein reducing the action potential conduction through the t-junction reduces symptoms of the sensory condition.

In some embodiments, applying the electric field to the dorsal root ganglion comprises positioning at least one electrode near the dorsal root ganglion, wherein the dorsal root has been explanted.

In a fourth aspect of the present invention, a system is provided for neuromodulation comprising at least one electrode positionable in proximity to a dorsal root ganglion, and a pulse generator electrically connectable with the at least one electrode, wherein the pulse generator provides an intermittent stimulation signal to the at least one electrode which creates an electric field which when applied to the dorsal root ganglion neuromodulates at least one subcellular structure on a primary sensory neuron within the dorsal root ganglion.

In some embodiments, the intermittent stimulation signal comprises a series of bursts and inter-burst delays, wherein the bursts have a frequency of up to approximately 1000 Hz. For example, in some embodiments the bursts have a frequency of approximately 4-1000 Hz.

In some embodiments, the intermittent stimulation signal comprises a series of bursts and inter-burst delays, wherein the bursts have a frequency of up to approximately 10,000 Hz.

In some embodiments, the intermittent stimulation signal comprises a series of bursts and inter-burst delays, wherein the inter-burst delays are approximately 4-1000 microseconds.

In some embodiments, the intermittent stimulation signal comprises a series of bursts and inter-burst delays, wherein the bursts are comprised of sine-waves. Alternatively, the bursts may be comprised of square waves.

In some embodiments, the at least one electrode is mounted on a lead, wherein the lead is configured to pass through an epidural space to position the at least one electrode in proximity to the dorsal root ganglion.

In some embodiments, the intermittent stimulation signal is configured to exclude stimulation of anatomy outside of the dorsal root ganglion. In some embodiments the intermittent stimulation signal is selective to subcellular structures.

Other objects and advantages of the present invention will become apparent from the detailed description to follow, together with the accompanying drawings.

### BRIEF DESCRIPTION OF THE DRAWINGS

FIG. 1 illustrates an embodiment of an implantable stimulation system.

FIG. 2 illustrates example placement of the leads of the embodiment of FIG. 1 within a patient anatomy.

FIG. 3 illustrates an example cross-sectional view of an individual spinal level showing a lead positioned on, near or about a target dorsal root ganglion.

FIG. 4A is a schematic illustration of a spinal cord, associated nerve roots, dorsal root ganglion and a peripheral nerve on a spinal level; FIG. 4B provides an expanded illustration of cells located in the DRG of FIG. 4A.

FIGS. 5A-5C is a cross-sectional histological illustration of a spinal cord and associated nerve roots, including a DRG.

FIGS. 6A-6D illustrate example embodiments of affecting the membranes of neurons within the dorsal root ganglion by at least one electric field generated by at least one electrode of a lead positioned in close proximity thereto.

FIG. 7A illustrates action potential conduction in its natural state while FIG. 7B illustrates the application of an electric field altering action potential conduction through a t-junction.

FIG. 8 schematically illustrates an example of an intermittent stimulation signal.

FIG. 9 illustrates an example in vitro model.

FIG. 10 illustrates a microscopic view of DRG neurons in situ.

FIG. 11 illustrates an example of measured intracellular Ca2+.

FIGS. 12A-12B illustrate example summary data.

FIG. 13 illustrates example anatomy.

FIGS. 14A-14B illustrate example sample traces.

FIGS. 15A-15B illustrate example summary data.

FIG. 16 illustrates example action potential generation in comparison to baseline.

FIG. 17A-17B illustrate example summary data.

### DETAILED DESCRIPTION

The present invention provides devices, systems and methods for the targeted treatment of abnormal sensory conditions, such as chronic pain, puritis, dysthesias and phantom limb pain. In such conditions, physical stimuli is transduced into neuronal impulses that are subsequently transmitted to the central nervous system for processing. Such transduction is achieved by primary sensory neurons in the dorsal root ganglions. Subcellular structures on primary sensory neurons can significantly modulate the function of these neurons, thereby affecting the transduction and reducing the abnormal sensory experiences. Thus, the present invention provides devices, systems and methods for neuromodulating subcellular structures on primary sensory neurons of the dorsal root ganglions. In most embodiments, neuromodulation comprises stimulation, however it may be appreciated that neuromodulation may include a variety of forms of altering or modulating nerve activity by delivering electrical and/or pharmaceutical agents directly to a target anatomy. For illustrative purposes, descriptions herein will be provided in terms of stimulation and stimulation parameters, however, it may be appreciated that such descriptions are not so limited and may include any form of neuromodulation and neuromodulation parameters.

The central nervous system includes the spinal cord and the pairs of nerves along the spinal cord which are known as spinal nerves. The spinal nerves include both dorsal and ventral roots which fuse to create a mixed nerve which is part of the peripheral nervous system. At least one dorsal root ganglion (DRG) is disposed along each dorsal root prior to the point of mixing. Thus, the neural tissue of the central nervous system is considered to include the dorsal root ganglions and exclude the portion of the nervous system beyond the dorsal root ganglions, such as the mixed nerves of the peripheral nervous system. Typically, the systems and devices of the present invention are used to stimulate one or more dorsal root ganglia, particularly, one or more subcellular structures of primary sensory neurons within the dorsal root ganglia, while minimizing or excluding undesired stimulation of other tissues, such as surrounding or nearby tissues outside of the dorsal root ganglia, ventral root and portions of the anatomy associated with body regions which are not targeted for treatment. However, it may be appreciated that stimulation of other tissues are contemplated.

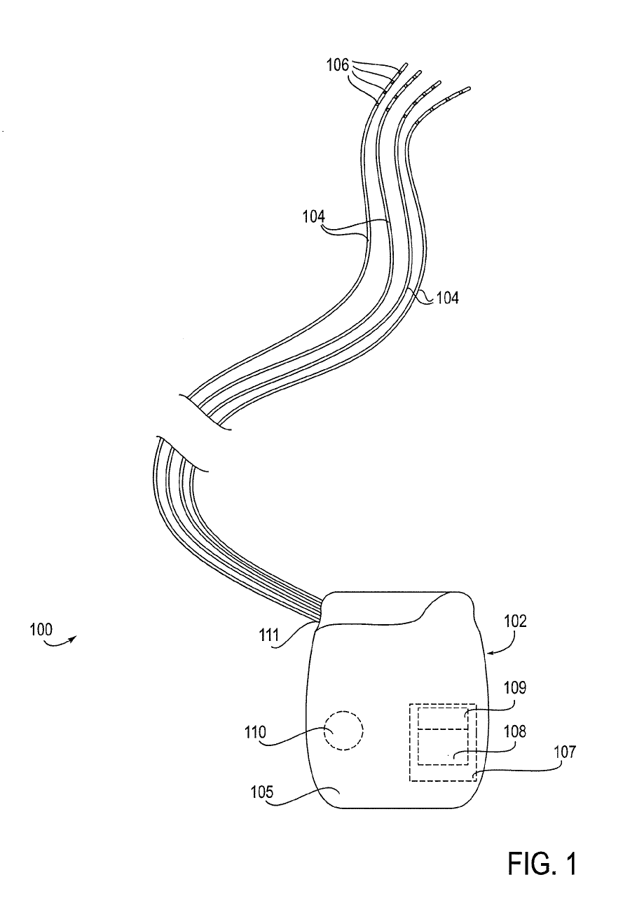

FIG. 1 illustrates an embodiment of an implantable stimulation system 100 for treatment of patients suffering from various sensory conditions. The system 100 includes an implantable pulse generator (IPG) 102 and at least one lead 104 connectable thereto. In preferred embodiments, the system 100 includes four leads 104, as shown, however any number of leads 104 may be used including one, two, three, four, five, six, seven, eight, up to 58 or more. Each lead 104 includes at least one electrode 106. In preferred embodiments, each lead 104 includes four electrodes 106, as shown, however any number of electrodes 106 may be used including one, two, three, four five, six, seven, eight, nine, ten, eleven, twelve, thirteen, fourteen, fifteen, sixteen or more. Each electrode can be configured as off, anode or cathode. In some embodiments, even though each lead and electrode are independently configurable, at any given time the software ensures only one lead is stimulating at any time. In other embodiments, more than one lead is stimulating at any time, or stimulation by the leads is staggered or overlapping.

Referring again to FIG. 1, the IPG 102 includes electronic circuitry 107 as well as a power supply 110, e.g., a battery, such as a rechargeable or non-rechargeable battery, so that once programmed and turned on, the IPG 102 can operate independently of external hardware. In some embodiments, the electronic circuitry 107 includes a processor 109 and programmable stimulation information in memory 108.

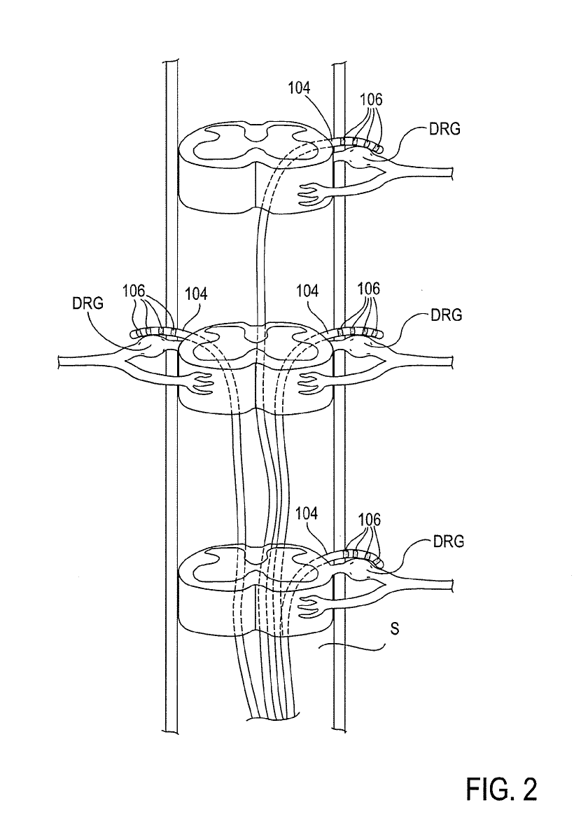

The implantable stimulation system 100 can be used to stimulate a variety of anatomical locations within a patient's body. In preferred embodiments, the system 100 is used to stimulate one or more dorsal root ganglions, particularly subcellular structures within primary sensory neurons of the dorsal root ganglions. FIG. 2 illustrates example placement of the leads 104 of the embodiment of FIG. 1 within the patient anatomy. In this example, each lead 104 is individually advanced within the spinal column S in an antegrade direction. Each lead 104 has a distal end which is guidable toward a target DRG and positionable so that its electrodes 106 are in proximity to the target DRG. Specifically, each lead 104 is positionable so that its electrodes 106 are able to selectively stimulate the DRG, either due to position, electrode configuration, electrode shape, electric field shape, stimulation signal parameters or a combination of these. FIG. 2 illustrates the stimulation of four DRGs, each DRG stimulated by one lead 104. These four DRGs are located on three levels, wherein two DRGs are stimulated on the same level. It may be appreciated that any number of DRGs and any combination of DRGs may be stimulated with the stimulation system 100 of the present invention. It may also be appreciated that more than one lead 104 may be positioned so as to stimulate an individual DRG and one lead 104 may be positioned so as to stimulate more than one DRG.

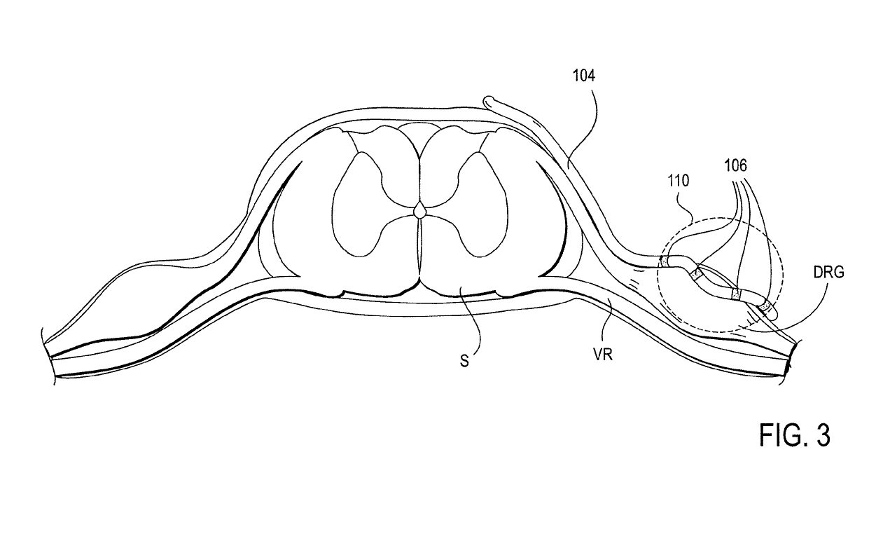

FIG. 3 illustrates an example cross-sectional view of an individual spinal level showing a lead 104 of the stimulation system 100 positioned on, near or about a target DRG. The lead 104 is advanced along the spinal cord S to the appropriate spinal level wherein the lead 104 is advanced laterally toward the target DRG. In some instances, the lead 104 is advanced through or partially through a foramen. At least one, some or all of the electrodes 106 are positioned on, about or in proximity to the DRG. In preferred embodiments, the lead 104 is positioned so that the electrodes 106 are disposed along a surface of the DRG opposite to the ventral root VR, as illustrated in FIG. 3. It may be appreciated that the surface of the DRG opposite the ventral root VR may be diametrically opposed to portions of the ventral root VR but is not so limited. Such a surface may reside along a variety of areas of the DRG which are separated from the ventral root VR by a distance.

In some instances, such electrodes 106 may provide a stimulation region indicated by dashed line 110, wherein the DRG receives stimulation energy within the stimulation region and the ventral root VR does not as it is outside of the stimulation region. Thus, such placement of the lead 104 may assist in reducing any possible stimulation of the ventral root VR due to distance. However, it may be appreciated that the electrodes 106 may be positioned in a variety of locations in relation to the DRG and may selectively stimulate the DRG due to factors other than or in addition to distance, such as due to stimulation profile shape and stimulation signal parameters, to name a few. It may also be appreciated that the target DRG may be approached by other methods, such as a retrograde epidural approach. Likewise, the DRG may be approached from outside of the spinal column wherein the lead 104 is advanced from a peripheral direction toward the spinal column, optionally passes through or partially through a foramen and is implanted so that at least some of the electrodes 106 are positioned on, about or in proximity to the DRG.

In order to position the lead 104 in such close proximity to the DRG, the lead 104 is appropriately sized and configured to maneuver through the anatomy. In some embodiments, such maneuvering includes atraumatic epidural advancement along the spinal cord S, through a sharp curve toward a DRG, and optionally through a foramen wherein the distal end of the lead 104 is configured to then reside in close proximity to a small target such as the DRG. Consequently, the lead 104 is significantly smaller and more easily maneuverable than conventional spinal cord stimulator leads. Example leads and delivery systems for delivering the leads to a target such as the DRG are provided in U.S. patent application Ser. No. 12/687,737, entitled “Stimulation Leads, Delivery Systems and Methods of Use”, incorporated herein by reference for all purposes.

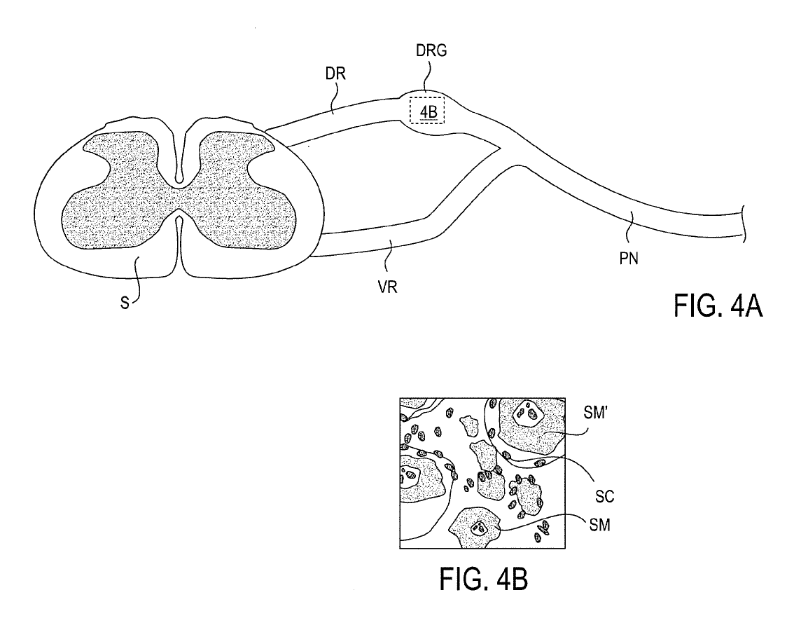

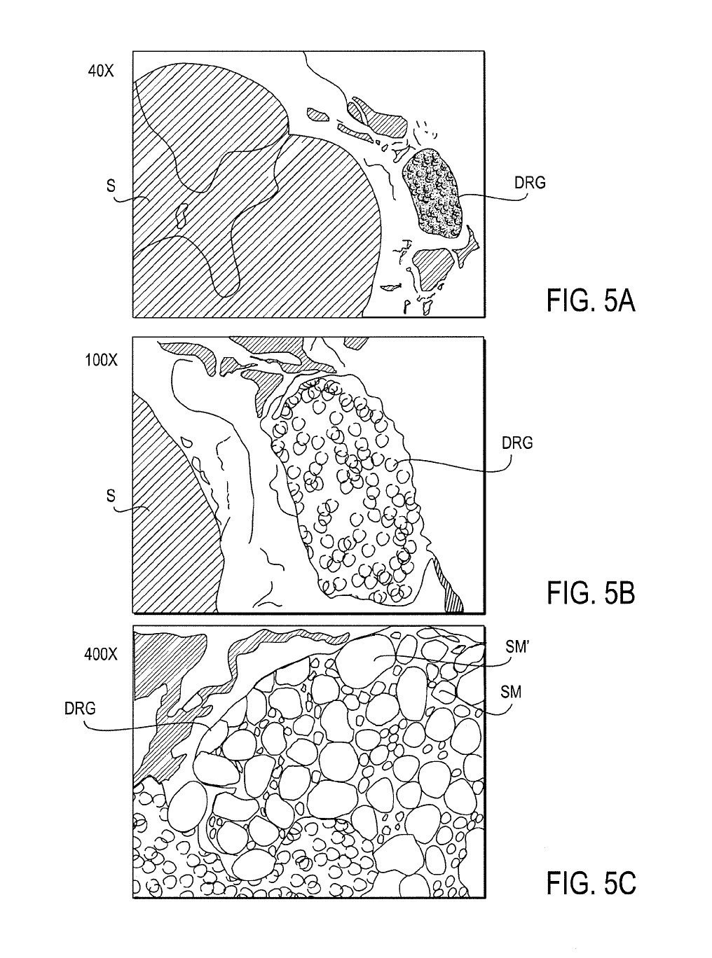

FIG. 4A provides a schematic illustration of a spinal cord S, associated nerve roots and a peripheral nerve on a spinal level. Here, the nerve roots include a dorsal root DR and a ventral root VR that join together at the peripheral nerve PN. The dorsal root DR includes a dorsal root ganglion DRG, as shown. The DRG is comprised of a variety of cells, including large neurons, small neurons and non-neuronal cells. Each neuron in the DRG is comprised of a bipolar or quasi-unipolar cell having a soma (the bulbous end of the neuron which contains the cell nucleus) and two axons. The word soma is Greek, meaning “body”; the soma of a neuron is often called the “cell body”. Somas are gathered within the DRG, rather than the dorsal root, and the associated axons extend therefrom into the dorsal root and toward the peripheral nervous system. FIG. 4B provides an expanded illustration of cells located in the DRG, including a small soma SM, a large soma SM′ and non-neuronal cells (in this instance, satellite cells SC). FIGS. 5A-5C provide a cross-sectional histological illustration of a spinal cord S and associated nerve roots, including a DRG. FIG. 5A illustrates the anatomy under 40× magnification and indicates the size relationship of the DRG to the surrounding anatomy. FIG. 5B illustrates the anatomy of FIG. 5A under 100× magnification. Here, the differing structure of the DRG is becoming visible. FIG. 5C illustrates the anatomy of FIG. 5A under 400× magnification focusing on the DRG. As shown, the larger soma SM′ and the smaller somas SM are located within the DRG.

All neurons are electrically excitable, maintaining voltage gradients across their membranes by means of metabolically driven ion pumps, which combine with ion channels embedded in the membrane to generate intracellular-versus-extracellular concentration differences of ions such as sodium, potassium, chloride, and calcium. Changes in the cross-membrane voltage can alter the function of voltage-dependent ion channels. If the voltage changes by a large enough amount, an all-or-none electrochemical pulse called an action potential is generated, which travels rapidly along the cell's axon, and activates synaptic connections with other cells when it arrives.

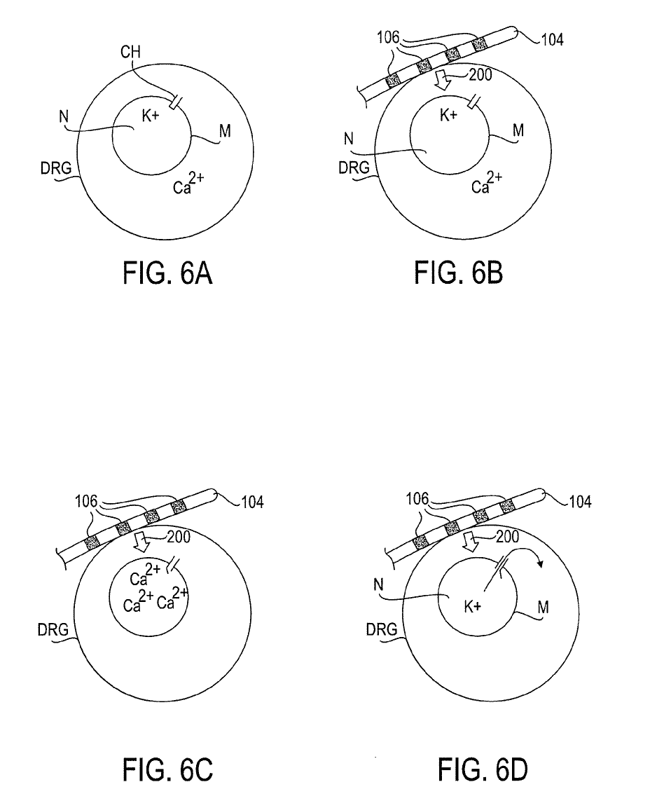

In some embodiments, the membranes of neurons within the dorsal root ganglion are affected by at least one electric field generated by at least one electrode 106 of the lead 104 positioned in close proximity thereto, as schematically illustrated in FIGS. 6A-6D. FIGS. 6A-6D schematically illustrate a dorsal root ganglion DRG having a neuron N. The neuron N has a membrane M which includes at least one potassium (K+) ion channel CH. Each potassium ion channel CH is dependent on calcium (Ca2+) to open the channel. FIG. 6A illustrates the neuron N in its natural state, wherein the potassium ion channels CH are closed. FIG. 6B illustrates the application of an electric field 200 provided by at least one electrode 106 on a lead 104 positioned in proximity to the DRG. The electric field 200 produces sufficient power to allow entry of calcium (Ca2+) into the neuron N. FIG. 6C schematically illustrates the increase of calcium (Ca2+) in the neuron N due to the electric field 200. The calcium (Ca2+) entry activates the calcium (Ca2+) dependent potassium (K+) ion channels CH, as illustrated in FIG. 6D. The potassium (K+) ion channels CH hyperpolarize the cell membrane M, making the neuron N less excitable. This will have the effect of membrane hypoexcitability and also reduce typical cellular firing characteristics, such as bursting or synchronized entrainment from sensory stimuli. Consequently, the patient will have reduced symptoms of their abnormal sensory conditions, such as reduced pain puritis, dysthesias, and/or phantom limb pain, to name a few.

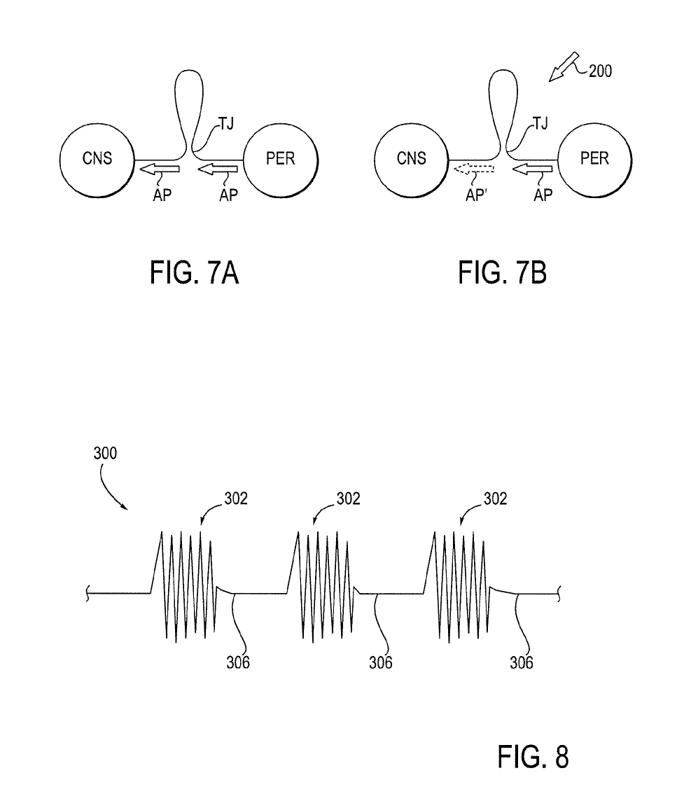

In other embodiments, subcellular structures other than ion channels are influenced by electric fields to affect functioning of primary sensory neurons. For example, in some embodiments, t-junctions are modulated. As mentioned, the soma or cell body of a primary sensory neuron resides in the dorsal root ganglion. The soma is attached midway along its axon by a short stem axon. The resulting t-shaped bifurcation is termed a “t-junction”, creating a pseudounipolar geometry. The t-junction forms where the axonal projection from the periphery (that sends action potentials from the periphery to the soma) and the axon of the primary sensory neuron (that sends action potentials from the soma to the spinal cord and brain) unify or meet. FIGS. 7A-7B schematically illustrate a t-junction TJ that connects the peripheral nervous system PER with the central nervous system CNS. FIG. 7A illustrates action potential conduction AP in its natural state. FIG. 7B illustrates the application of an electric field 200 (generated by at least one electrode 106 of the lead 104 positioned in close proximity to the DRG), altering action potential conduction AP′ through the t-junction TJ, such as from the t-junction to the central nervous system CNS. Such alteration of action potentials alters sensory stimuli to the central nervous system. Thus, the t-junction can act as a filter to disallow the transduction of undesired sensory information. When the patient suffers from abnormal sensory conditions, such as chronic pain, puritis, dysthesias and phantom limb pain, the abnormal sensory stimuli causing these conditions are blocked or altered so as to reduce the symptoms and treat the condition.

In some embodiments, selective stimulation of the involved sensory neuron SN and subcellular structures is achieved with the choice of the size of the electrode(s), the shape of the electrode(s), the position of the electrode(s), the stimulation signal, pattern or algorithm, or any combination of these. Such selective stimulation stimulates the targeted neural tissue while excluding untargeted tissue, such as surrounding or nearby tissue. In some embodiments, the stimulation energy is delivered to the targeted neural tissue so that the energy dissipates or attenuates beyond the targeted tissue or region to a level insufficient to stimulate modulate or influence such untargeted tissue. In particular, selective stimulation of tissues, such as the DRG or portions thereof, exclude stimulation of the ventral root wherein the stimulation signal has an energy below an energy threshold for stimulating a ventral root associated with the target dorsal root while the lead is so positioned. Examples of methods and devices to achieve such selective stimulation of the DRG are provided in U.S. patent application Ser. No. 12/607,009, entitled “Selective Stimulation Systems and Signal Parameters for Medical Conditions”, incorporated herein by reference for all purposes.

In some embodiments, stimulation of the involved subcellular structures of the sensory neuron SN is achieved by an intermittent stimulation signal provided to the at least one electrode 106 of the lead 104. An example of such an intermittent stimulation signal 300 is schematically illustrated in FIG. 8. Here the signal 300 is comprised of a series of bursts 302 separated by inter-burst delays 304. It may be appreciated that the bursts 302 may be comprised of one or more different types of waves, such as sine-waves or square waves. In some embodiments, the bursts 302 have a frequency of up to approximately 1000 Hz, such as approximately 4-1000 Hz. In other embodiments, the bursts 302 have frequency of 1000-2000 Hz, 2000-3000 Hz, 3000-4000 Hz, 4000-5000 Hz, 5000-6000 Hz, 6000-7000 Hz, 7000-8000 Hz, 8000-9000 Hz, or 9000-10,000 Hz. In some embodiments, the inter-burst delays 304 are approximately 4-1000 microseconds. These frequencies and inter-burst intervals maximize the duty cycle and minimize the actual power delivered to the tissues. Neurons are highly responsive to pulsatile stimuli and the membrane and intracellular effects are amplified when utilizing these stimulation parameters.

Typically, the intermittent stimulation signal is configured to exclude stimulation of anatomy outside of the dorsal root ganglion, such as nearby tissues, particularly including the ventral root associated with the dorsal root ganglion. In some embodiments, the intermittent stimulation signal is selective to subcellular structures. In some instances, the stimulation signal stimulates the subcellular structures while excluding or minimizing stimulation of other structures within the dorsal root ganglion.

In vitro studies were undertaken to confirm the alterations in cellular mechanisms within the dorsal root ganglion when affected by externally applied electrical fields, such as those applied to dorsal root ganglions in vivo according to the systems, devices and methods described herein. An example of such an in vitro study is as follows:

### Methods

Subjects: male Sprague-Dawley rats (150-175 g at the initiation of the protocol). All procedures were approved by the MCW IACUC.

Tissue Preparation: Intact DRGs were harvested from anesthetized animals and bathed in artificial CSF: NaCl 128, KCl 3.5, MgCl2 1.2, CaCl2 2.3, NaH2PO4 1.2, NaHCO3 24.0, glucose 11) bubbled by 5% CO2 and 95% O2 to maintain a pH of 7.35. Electrodes (60-90 MΩ) were filled with 2M K+ acetate buffered with 10 mM HEPES.

Neuronal Activation: Somatic action potentials (APs) were generated in one of 2 ways. A) For Experiment 1, axons in the dorsal root were depolarized by bipolar stimulation, whereby APs were conducted to the neuronal soma. B) For Experiment 2, direct membrane depolarization of the soma was achieved by current injection through the recording electrode, for which voltage error was minimized using a discontinuous current clamp mode with a switching rate of 2 kHz.

Electrophysiological Recording: During impalement, tissue was observed using differential interference contrast microscopy with infrared illumination. Somata were selected with diameters <35 μm. Recording was initiated only after the resting membrane potential RMP had stabilized (<1 min) and only if RMP <−45 mV. Neurons were assigned to control or treatment groups randomly.

Electrical DRG Treatment: The electrical stimulation device was programmed to deliver pulses of 400 μs duration and 60 Hz continuously during the 90 s treatment period. Stimulus voltage was monitored online by oscilloscope. Each DRG received only a single electrical stimulation treatment.

Experiment 1: AP trains initiated by axonal stimulation were delivered at frequencies of 10, 50, and 100 Hz (in that order), with a 10 s interval between, while recording their conduction into the soma. Test trains after electrical treatment began following a 5 s delay. Controls received no electrical treatment but also had a 95 s delay between test trains. The effect of electrical treatment on success rate for conducting APs into the neuronal soma was compared to the effect of time alone in control neurons.

Experiment 2: Depolarization current (100 ms) was injected through the recording electrode in amplitudes that increased by 0.2 nA increments separated by 2 s intervals. Firing patterns induced by depolarization were recorded simultaneously. Electrical treatment of the DRG (or comparable time without treatment) was followed by a similar sequence of steps. The effect of electrical treatment on the number of APs generated by depolarization was compared to the effect of time alone in control neurons.

Statistics: Data are shown for mean±SEM.

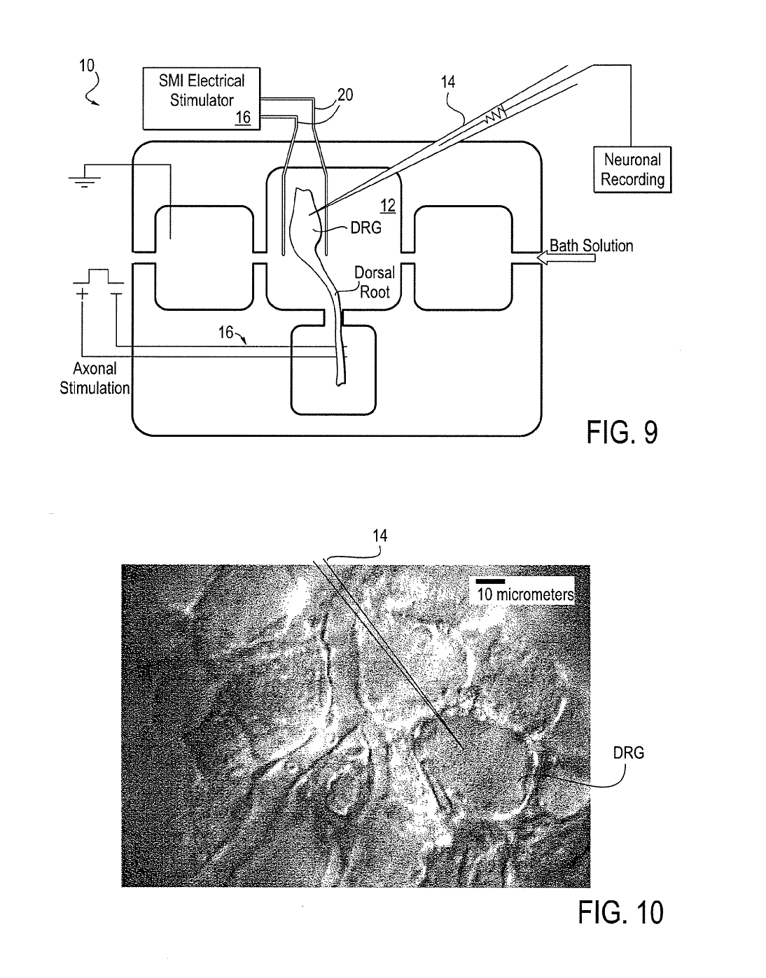

Referring to FIG. 9, an in vitro model 10 was devised for recording neuronal membrane events during field stimulation of dorsal root ganglia (DRGs). DRG excised from adult rats were placed in a custom chamber 12 perfused with oxygenated artificial CSF at 37° C. Sharp electrode impalement provided trans-membrane potential (Vm). Neuronal activation was produced by direct depolarization through the recording electrode 14 or by conduced action potentials (APs) initiated by axonal stimulation 16. A pulse generator or electrical stimulator 16 discharges on either side of the DRG through platinum electrodes 20 to produce fields that resemble a clinical device. Example electrical stimulators are provided in PCT Patent Application No. PCT/US2005/031960 entitled, “Neurostimulation Methods and Systems” and U.S. patent application Ser. No. 12/607,009, entitled, “Selective Stimulation Systems and Signal Parameters for Medical Conditions”, both of which are incorporated by reference for all purposes.

FIG. 10 provides a microscopic view of DRG neurons in situ with a scaled representation of the recording electrode 14 superimposed. This technique preserves DRG structure and minimally affects cytoplasmic signaling.

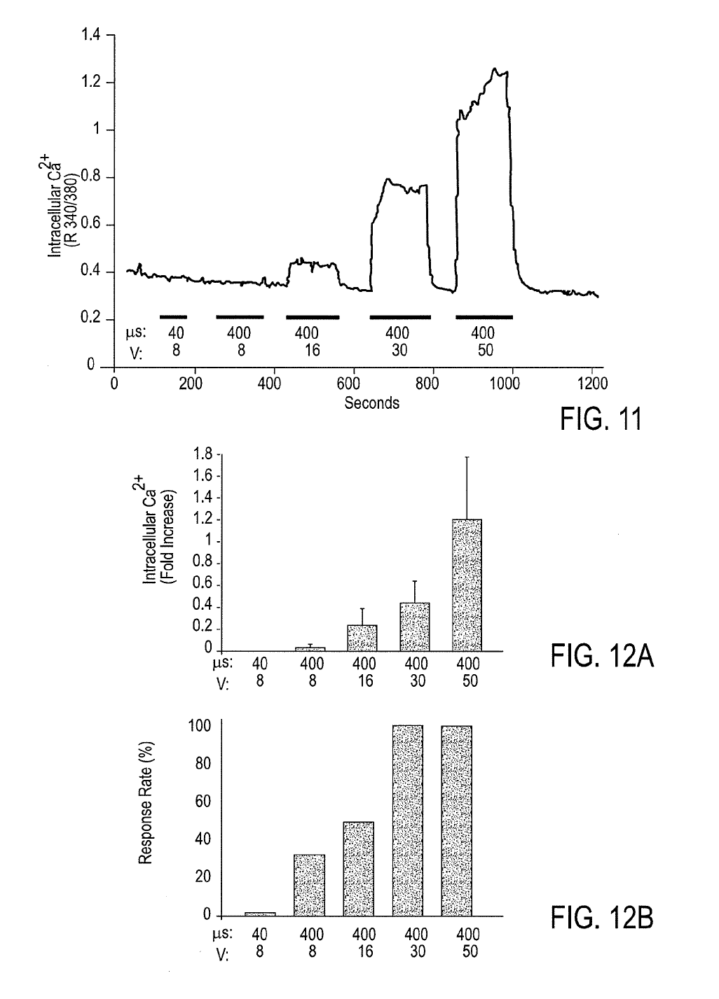

In the in vitro model, the field stimulator discharges through a high load (saline bath solution), unlike in vivo conditions. To match the neuronal effects, we determined the pulse parameters needed to activate the DRG neurons, as is noted in humans (sensation of paresthesias). DRG neurons admit Ca2+ when active, which we measured with intracellular Fura-2 by microfluorimetry (as illustrated in FIG. 11). Thus, relevant stimulation parameters were designed to replicated clinical conditions.

Referring to FIGS. 12A-12B, in 6 neurons, cytoplasmic Ca2+ increase showed a dependence upon stimulation intensity. 30V and pulse duration of 400 μs produced activation of all neurons. These parameters were used for subsequent experiments.

In the first experiment, electrical DRG stimulation increases impulse filtering at the T-junction of sensory neurons in the DRG.

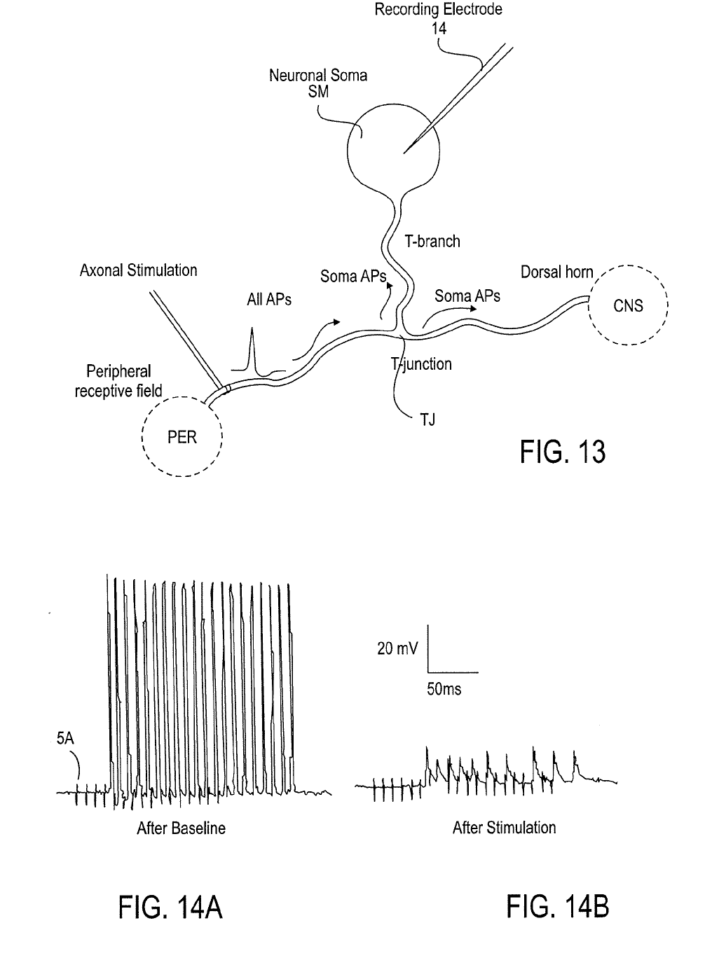

Referring to FIG. 13, afferent APs initiated in the peripheral receptive field propagate proximally, but conduction may fail at points of impedance mismatch, particularly the T-junction. AP arrival was monitored in the soma to identify successful conduction to the dorsal root and dorsal horn of the cord.

Referring to FIG. 14A-14B, sample traces showing 100Hz axonal stimulation, with no conduction failure at baseline but failed AP invasion of the T-branch by (*) following electrical stimulation. APs have reduced amplitude after stimulation due to additional conduction failure at the junction of the T-branch and soma.

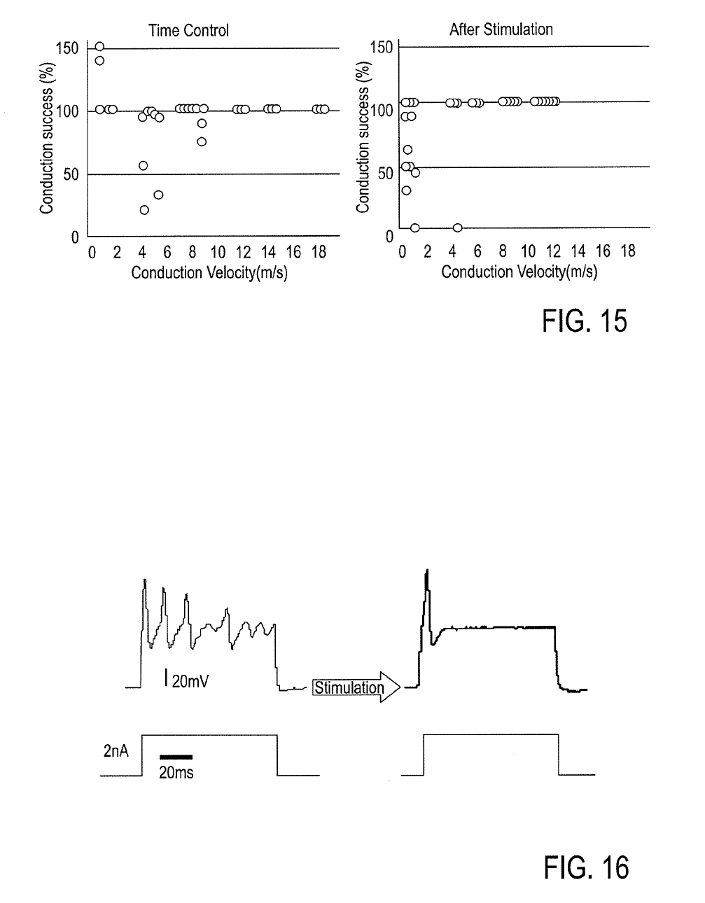

Referring to FIGS. 15A-15B, summary data shows increased failure of conduction through the T-junction after electrical field stimulation. Regression analysis showed a significant effect of conduction velocity CV on conduction success only after stimulation. Also, conduction success is significantly lower after stimulation vs. time control for units with CV <5 m/s (presumed nociceptors).

In the second experiment, electrical DRG stimulation inhibits sensory neuron impulse.

Referring to FIG. 16, AP generation during neuronal depolarization (by current injected via the recording electrode) was compared to baseline in repetitively firing neurons after either field stimulation or comparable time without stimulation (control).

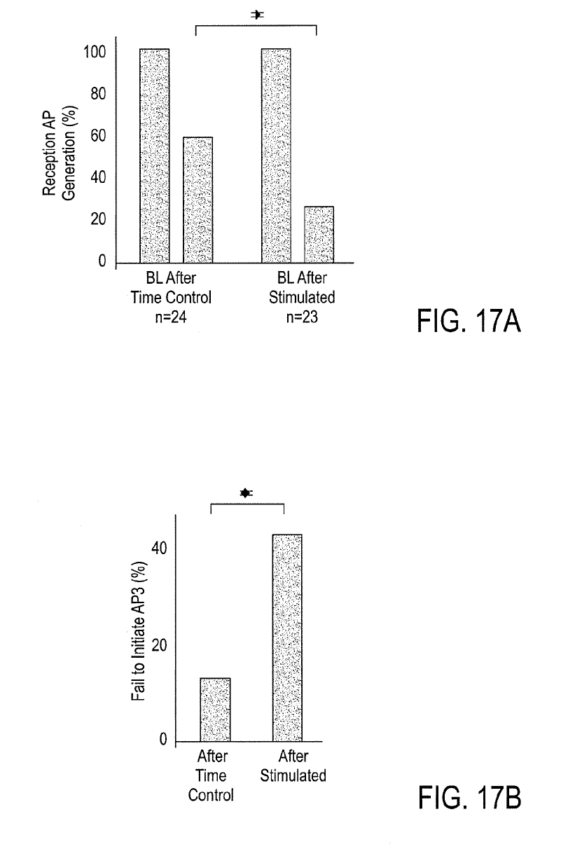

Referring to FIGS. 17A-17B, summary data shows a significant decrease in the ability of DRG neurons to fire repetitively (left) or to initiate the first stimuli upon depolarization (right), after electrical stimulation. (The falloff in repetitive firing after time control alone is due to the effect of the neural activity induced during baseline depolarization.)

### Conclusions

1) Electrical field stimulation of the DRG in vitro inhibits conduction of trains of APs through the neuronal T-junction, with a preferential effect on slow-conducting nociceptive units.

2) Electrical field stimulation also suppresses initiation of AP firing in sensory neurons.

3) The mechanism of both of these processes may involve accumulation of cytoplasmic Ca2+.

4) These phenomena may contribute to a peripheral mechanism of analgesia following therapeutic stimulation of the DRG.

As mentioned previously, it may be appreciated that neuromodulation may include a variety of forms of altering or modulating nerve activity by delivering electrical and/or pharmaceutical agents directly to a target area. For illustrative purposes, descriptions herein were provided in terms of stimulation and stimulation parameters, however, it may be appreciated that such descriptions are not so limited and may include any form of neuromodulation and neuromodulation parameters, particularly delivery of agents to the dorsal root ganglion. Methods, devices and agents for such delivery are further described in U.S. patent application Ser. No. 13/309,429 entitled, “Directed Delivery of Agents to Neural Anatomy”, incorporated herein by reference.

Although the foregoing invention has been described in some detail by way of illustration and example, for purposes of clarity of understanding, it will be obvious that various alternatives, modifications, and equivalents may be used and the above description should not be taken as limiting in scope of the invention which is defined by the appended claims.

What is claimed is:

1. A method of neuromodulation comprising:
positioning at least one electrode in proximity to a dorsal root ganglion; and
energizing the at least one electrode so that an electric field is applied to the dorsal root ganglion in a manner which neuromodulates at least one subcellular structure on a primary sensory neuron within the dorsal root ganglion.

2. A method as in claim 1, wherein neuromodulating the at least one subcellular structure comprises hyperpolarizing a cell membrane of the primary sensory neuron.

3. A method as in claim 1 or 2, wherein the subcellular structure comprises an ion channel of a cell membrane of the primary sensory neuron.

4. A method as in claim 3, wherein the ion channel comprises a potassium ion channel.

5. A method as in any of the above claims, wherein neuromodulating the at least one subcellular structure comprises reducing cellular firing characteristics of the primary sensory neuron.

6. A method as in any of the above claims, wherein the dorsal root ganglion is associated with an abnormal sensory condition of a patient and wherein neuromodulating the at least one subcellular structure reduces a symptom of the sensory condition.

7. A method as in claim 6, wherein the abnormal sensory condition comprises pain, puritis, dysthesias, phantom limb pain or a combination of these.

8. A method as in any of claims 1-5, wherein the dorsal root ganglion is disposed within an in vitro model, and further comprising measuring an effect of the electric field on membrane excitability of the primary sensory neuron.

9. A method as in claim 8, wherein the measured effect indicates decreased membrane excitability.

10. A method as in claim 1, wherein neuromodulating the at least one subcellular structure comprises modulating at least one t-junction.

11. A method as in claim 10, wherein modulating the at least one t-junction comprises altering action potential conduction through the at least one t-junction.

12. A method as in claims 10 or 11, wherein the dorsal root ganglion is disposed within an in vitro model, and further comprising measuring amplitude of at least one train of action potentials through the at least one t-junction during and/or after neuromodulation.

13. A method as in claim 12, wherein measuring comprises measuring a reduction in amplitude.

14. A method as in claim 12, wherein measuring comprises measuring a decrease in bursting behavior of the neuron associated with the t-junction.

15. A method as in any of the above claims, energizing the at least one electrode comprise providing an intermittent stimulation signal comprised of a series of bursts and inter-burst delays.

16. A method as in claim 15, wherein the bursts have a frequency of approximately 4-1000 Hz.

17. A method as in claim 15, wherein the inter-burst delays are approximately 4-1000 microseconds.

18. A method of reducing excitability of a neuron within a dorsal root ganglion, comprising:
applying an electric field to the dorsal root ganglion, wherein the electric field produces sufficient power to allow entry of calcium into the neuron to at least a level which activates calcium dependent potassium ion channels,
whereby the potassium ion channels hyperpolarize the cell membrane making the neuron less excitable.

19. A method as in claim 18, wherein applying the electric field to the dorsal root ganglion comprises positioning a lead having at least one electrode in proximity to the dorsal root ganglion within a patient so that at least one electrode provides the electric field.

20. A method as in claim 19, wherein positioning the lead comprises advancing the lead within an epidural space of the patient.

21. A method as in claim 18, 19 or 20, wherein the dorsal root ganglion is associated with an abnormal sensory condition of a patient and wherein making the neuron less excitable reduces symptoms of the sensory condition.

22. A method as in claim 18, 19 or 20 wherein applying the electric field to the dorsal root ganglion comprises positioning at least one electrode near the dorsal root ganglion, wherein the dorsal root has been explanted.

23. A method of suppressing action potential firing in a sensory neuron within a dorsal root ganglion, comprising:
applying an electric field to the dorsal root ganglion so that the electric field neuromodulates a t-junction associated with the sensory neuron in a manner which reduces action potential conduction through the t-junction.

24. A method as in claim 23, wherein applying the electric field to the dorsal root ganglion comprises positioning a lead having at least one electrode in proximity to the dorsal root ganglion within a patient so that at least one electrode provides the electric field.

25. A method as in claim 24, wherein positioning the lead comprises advancing the lead within an epidural space of the patient.

26. A method as in claim 23, 24 or 25, wherein the dorsal root ganglion is associated with an abnormal sensory condition of the patient and wherein reducing the action potential conduction through the t-junction reduces symptoms of the sensory condition.

27. A method as in claim 23, wherein applying the electric field to the dorsal root ganglion comprises positioning at least one electrode near the dorsal root ganglion, wherein the dorsal root has been explanted.

28. A system for neuromodulation comprising:
at least one electrode positionable in proximity to a dorsal root ganglion; and
a pulse generator electrically connectable with the at least one electrode, wherein the pulse generator provides an intermittent stimulation signal to the at least one electrode which creates an electric field which when applied to the dorsal root ganglion neuromodulates at least one subcellular structure on a primary sensory neuron within the dorsal root ganglion.

29. A system as in claim 28, wherein the intermittent stimulation signal comprises a series of bursts and inter-burst delays, wherein the bursts have a frequency of up to approximately 1000 Hz.

30. A system as in claim 29, wherein the bursts have a frequency of approximately 4-1000 Hz.

31. A system as in claim 28, wherein the intermittent stimulation signal comprises a series of bursts and inter-burst delays, wherein the bursts have a frequency of up to approximately 10,000 Hz.

32. A system as in any of claims 28-31, wherein the intermittent stimulation signal comprises a series of bursts and inter-burst delays, wherein the inter-burst delays are approximately 4-1000 microseconds.

33. A system as in any of claims 28-32, wherein the intermittent stimulation signal comprises a series of bursts and inter-burst delays, wherein the bursts are comprised of sine-waves.

34. A system as in any of claims 28-32, wherein the intermittent stimulation signal comprises a series of bursts and inter-burst delays, wherein the bursts are comprised of square waves.

35. A system as in any of claims 28-34, wherein the at least one electrode is mounted on a lead, wherein the lead is configured to pass through an epidural space to position the at least one electrode in proximity to the dorsal root ganglion.

36. A system as in any of claims 28-35, wherein the intermittent stimulation signal is configured to exclude stimulation of anatomy outside of the dorsal root ganglion.

37. A system as in claim 36, wherein the intermittent stimulation signal is selective to subcellular structures.
